(19) United States
(12) Patent Application Publication
(10) Pub. No.: US 2019/0380844 A1
(43) Pub. Date: Dec. 19, 2019

(54) SURGICAL PROBE DEVICE

(71) Applicant: Xialing Zhang, Mountain View, CA (US)

(72) Inventor: Xialing Zhang, Mountain View, CA (US)

(21) Appl. No.: 16/400,957

(22) Filed: May 1, 2019

Related U.S. Application Data
(60) Provisional application No. 62/686,069, filed on Jun. 17, 2018.

(51) Int. Cl. A61F 2/46; A61B 5/04; A61B 34/20; A61N 1/05; A61B 90/00

(52) U.S. Cl. CPC A61F 2/4611; A61B 5/04001; A61B 34/20; A61B 2034/2053; A61B 90/06; A61B 2090/062; A61N 1/0551

(57) ABSTRACT

A probe device is adapted for use in enabling implanted articles of spine surgery to be accurately and safely positioned relative to anatomy that can be injured during placement of such implanted article. The probe device can be used with a neuromonitoring system to assist in location of the spinal nerves by administration of low-voltage electrical stimulation during bone preparation and/or placement (e.g., insertion) of an implanted article (e.g., a bone screw) during an open or percutaneous minimally-invasive surgical approach of the spine. By monitoring the low-voltage electrical stimulation and/or a signal generated in response the low-voltage electrical stimulation being the administered, proximity to a patient's anatomy that is susceptible to being injured by the bone preparation and/or placement of the implanted article can be determined and placement of the implanted article can be performed in a manner that minimizes the potential for injury to such anatomy.

### CROSS-REFERENCE TO RELATED APPLICATIONS

This non-provisional utility patent application claims priority from co-pending U.S. Provisional Patent Application having Ser. No. 62/686,069, filed 17-Jun. 2018, entitled “EMG Spine Guiding Probe”, having a common applicant herewith and being incorporated herein in its entirety by reference.

### FIELD OF THE DISCLOSURE

The disclosures made herein relate generally to devices, apparatuses and methods for surgical procedures and, more particularly, a neuromonitoring probe device for aiding in the guidance of surgical tools and implanted articles during surgery.

### BACKGROUND

Spine surgery procedures of various known types have become commonplace. Whether these surgeries are performed in an open or minimally-invasive manner, they generally involve placement of one or more implanted articles within or on one or more bony structures of the spine. For example, vertebrae fixation is a common type of spine surgery in which adjacent vertebrae are fixed relative to each other through use of one or more implanted articles (e.g., a set of bone screws, plates and/or interbody cages).

It is well known that during the spine surgeries placement of implanted articles has risk of injury of anatomy that can be injured (e.g., spinal cord and spine nerve roots). As a result, surgical procedures for placement of implanted articles and equipment used in these surgeries must minimizes the potential for such injury. One important consideration in minimizing the potential for such injury is the surgical procedure being performed in a manner that provides for accurately positioning the implanted articles relative to anatomy that can be injured.

Therefore, surgical tools that provide for implanted articles to be accurately and safely positioned relative to anatomy that can be injured during placement of such implanted articles are advantageous, desirable and useful.

### SUMMARY OF THE DISCLOSURE

Embodiments of the present invention are directed to a probe device adapted for use in enabling implanted articles of spine surgery to be accurately and safely positioned relative to anatomy that can be injured during placement of such implanted article. More specifically, embodiments of the present invention are directed to a probe device that can be used with a neuromonitoring system to assist in determining location of anatomy susceptible to injury. Determining the location preferably includes administration (e.g., selective output) of a low-voltage electrical stimulation during bone preparation and/or placement (e.g., insertion) of an implanted article (e.g., a bone screw) such as during an open or percutaneous minimally-invasive spine surgery. By monitoring the low-voltage electrical stimulation and/or a signal generated in response the low-voltage electrical stimulation being the administered, proximity to a patient's anatomy that is susceptible to being injured by the bone preparation and/or placement of the implanted article can be determined and placement of the implanted article can be performed in a manner that minimizes the potential for injury to such anatomy.

In one embodiment of the present invention, a probe device comprises a first handle assembly and a second handle assembly. The first handle assembly comprises a first handle body and a stimulation probe. The stimulation probe has opposing end portions. A first one of the opposing end portions of the stimulation probe is attached to the first handle body such that the stimulation probe protrudes away from the first handle body. The second handle assembly comprises a second handle body and a probe tube. The probe tube has opposing end portions. A first one of the end portions of the probe tube is attached to the second handle body such that the probe tube protrudes away from the second handle body. A central passage of the probe tube is accessible through a stimulation probe receiving passage of the second handle body. A central portion of the stimulation probe between the opposing end portion thereof is disposed within the central passage of the probe tube such that a first one of the opposing end portions of the stimulation probe is positioned adjacent to the second handle body and a second one of the opposing end portions of the stimulation probe is positioned adjacent to a second one of the opposing end portions of the probe tube. The central portion of the stimulation probe is translatably disposed within the central passage of the probe tube such that the stimulation probe can be translated along a length of the probe tube for enabling the first handle assembly to be detached from the second handle assembly.

In another embodiment of the present invention, a probe device comprises a handle, a probe tube and a stimulation probe. The handle can include a lower handle body and an upper handle body. The probe tube has opposing end portions. A first one of the end portions of the probe tube is attached to the lower handle body such that the probe tube protrudes therefrom. A central passage of the probe tube intersects a stimulation probe receiving passage of the lower handle body. The stimulation probe has opposing end portions and a central portion extending therebetween. The stimulation probe is made from an electrically conductive material. A first one of the opposing end portions is attached to the upper handle body. The central portion of the stimulation probe is slideably disposed within the central passage of the probe tube for enabling the stimulation probe to be removed from therein by axially translating the stimulation probe relative to the probe tube. A second one of the opposing end portion of the stimulation probe is located adjacent to a second one of the opposing end portions of the probe tube and wherein electrically insulating material is provided within the central passage between the central portion of the stimulation probe and the probe tube. The first handle body and second handle body include mating engagement members that engage each other when the first handle body is in a seated position on the second handle body. Unrestricted relative rotation movement in at least one rotational direction between the first handle body and the second handle body and/or unrestricted relative axial movement between the first handle body and the second handle body is inhibited when the mating engagement members thereof are engaged with each other.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a perspective view showing a probe device in accordance with an embodiment of the present invention, wherein the first handle body is in a seated position relative to the second handle body.

FIG. 2 is a perspective view showing the probe device of FIG. 1 with handle bodies thereof in a separated position.

FIG. 3 is a cross-sectional view taken along the line 3-3 in FIG. 1.

### DETAILED DESCRIPTION

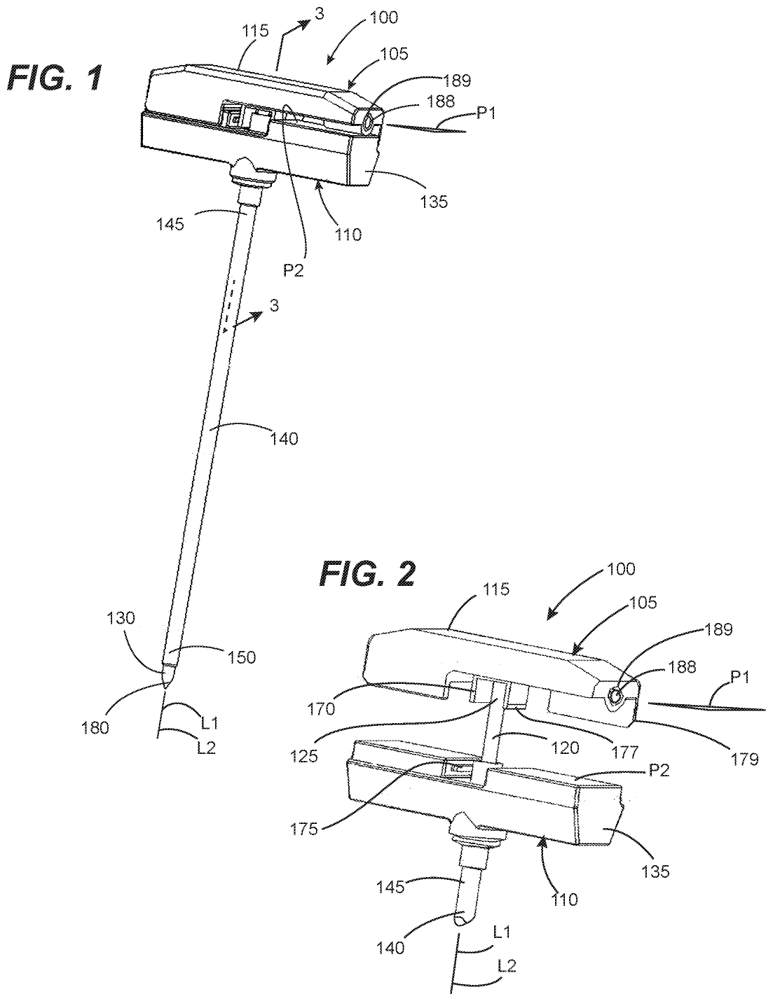

Referring to FIGS. 1-3, a probe device 100 configured in accordance with an embodiment of the present invention is adapted for use in enabling implanted articles of spine surgery to be accurately positioned relative to anatomy that can be injured during placement of such implanted article. The probe device 100 can be used with a neuromonitoring system (i.e., a neuromonitoring probe device) to assist in location of the spinal nerves by administration of low voltage electrical stimulation during bone preparation and/or placement (e.g., insertion) of an implanted article (e.g., a bone screw) during an open or percutaneous minimally-invasive surgical approach of the spine. By monitoring a signal generated in response the low voltage electrical stimulation being the administered, proximity to a patient's anatomy that is susceptible to being injured by the bone preparation and/or placement of the implanted article can be determined and affected to minimizes the potential for injury to such portion of the patient's anatomy.

The probe device 100 can comprise a first handle assembly 105 and a second handle assembly 110. The first handle assembly can comprise a first handle body 115 (i.e., an upper handle body) and a stimulation probe 120. The stimulation probe 120 has opposing end portions 125, 130 and is preferably elongated so as to be of a length sufficient to extend over the distance of a surgical site (e.g., about 30 cm or more). A first one of the opposing end portions (i.e., first end portion 125) of the stimulation probe 120 is attached to the first handle body 115 in a manner such that the stimulation probe 120 protrudes away from the first handle body 115. Preferably, the stimulation probe 120 protrudes away from the first handle body 115 such that a longitudinal axis L1 of the stimulation probe 120 extends generally perpendicular to a major horizontal planar surface P1 of the first handle body 115.

The second handle assembly can comprise a second handle body 135 (i.e., a lower handle body) and a probe tube 140. Jointly, the first handle body 115 and the second handle body 135 can be integrated or otherwise coupled to each other to define a one-piece or multi-piece handle of a probe device configured in accordance with an embodiment of the present invention. In preferred embodiments, such a one-piece or multi-piece handle can be configured to provide the same functionality as the two-piece handle shown and discussed in reference to FIGS. 1-3.

The probe tube 140 has opposing end portions 145, 150 and is preferably elongated so as to be of a length sufficient to extend over the distance of a surgical site (e.g., about 30 cm or more). A first one of the end portions of the probe tube (i.e., a first end portion 145) is attached to the second handle body 135 such that the probe tube 140 protrudes away from the second handle body 135. Preferably, the probe tube 140 protrudes away from the second handle body 135 such that a longitudinal axis L2 of the probe tube 140 extends generally perpendicular to a major horizontal planar surface P2 of the second handle body 135. Major horizontal planar surface P2 of the second handle body 135 preferably, but not necessarily, extends parallel to the major horizontal planar surface P1 of the first handle body 115.

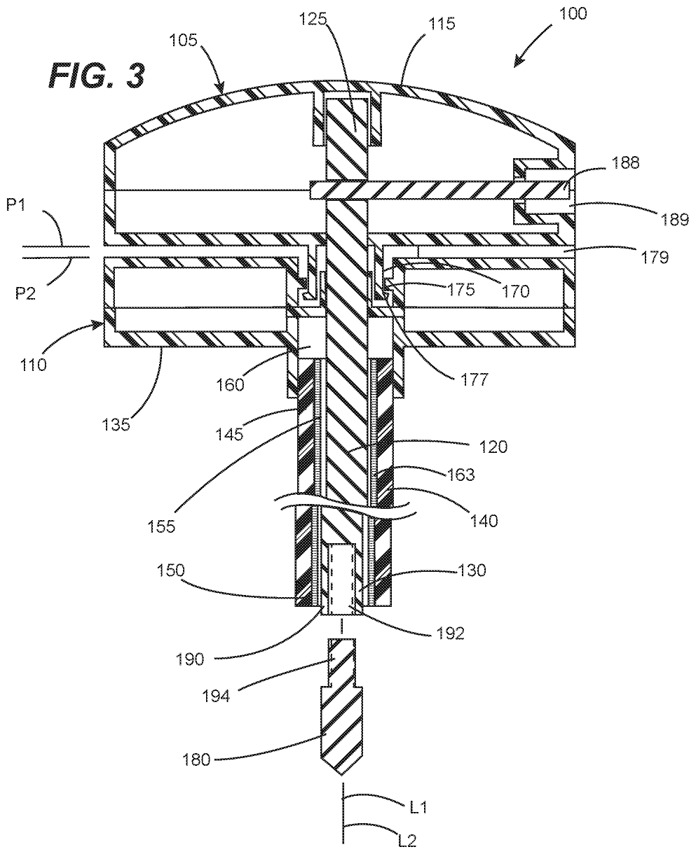

A central passage 155 of the probe tube 140 is accessible through a stimulation probe receiving passage 160 of the second handle body, as best shown in FIG. 3. A central portion 165 of the stimulation probe 120 between the opposing end portion 125, 130 thereof is disposed within the central passage 155 of the probe tube 140. Preferably, the stimulation probe 120 can be translatably (e.g., slidably) and rotatably engaged within the central passage 155 of the probe tube 140. To this end, preferably, the stimulation probe 120 and the probe tube 140 preferably both have a round cross-sectional shape, the stimulation probe 120 is coaxially disposed within the central passage 155 of the probe tube 140 and a centerline longitudinal axis L3 of the central passage of the probe tube extends along a straight reference axis upon which the longitudinal axes L1, L2 of the stimulation probe 120 and probe tube 140 lie. Lengths of the stimulation probe 120 and the probe tube 140 are preferably such that the first end portion 125 of the stimulation probe 120 is positioned adjacent to the stimulation probe receiving passage 160 of the second handle body 135 and a second one of the opposing end portions of the stimulation probe (i.e., the second end portion 130) is positioned adjacent to a second one of the opposing end portions (the second end portion 150) of the probe tube 140 (i.e., when the first handle body 115 is in the seated position on the second handle body 135, as shown in FIG. 1).

In use, it is desirable for the first handle assembly 105 to be separated from the second handle assembly 110 by applying opposing axial forces on the handle bodies for causing translation of the stimulation probe relative to the probe tube 140 until the stimulation probe 120 is extracted from within the central passage 155 of the probe tube 140. Such action inhibits (i.e., limiting to a controlled degree) unrestricted relative translation between the first and second handle bodies 115, 135. It is also desirable for unrestricted relative rotational movement between the first and second handle bodies to be inhibited (i.e., at least in one rotational direction).

Unrestricted movement generally refers to there being no mechanical structure that precludes unintentional movement to a degree that affects a given functionality of the device. To limit such unrestricted relative movements, the first and second handle bodies 115, 135 are preferably jointly configured for inhibiting and enabling relative rotational and translational movement between the stimulation probe 120 and the probe tube 140. For example, as best shown in FIGS. 2 and 3, the first handle body 115 and the second handle body 135 include first mating engagement members 170, 175 that engage each other when the first handle body 115 is in a seated position (i.e., position shown in FIG. 1) on the second handle body 135. Preferably, as shown, the first mating engagement members 170, 175 are configured for inhibiting relative rotation movement in at least one rotational direction between the first handle body 115 and the second handle body 135 and axial movement between the first handle body 115 and the second handle body 135 when the first mating engagement members 170, 175 are engaged with each other (i.e., when the first handle body 115 is in a seated position on the second handle body 135, as shown in FIG. 1). The planar flat shape of the first mating engagement members 170, 175 inhibit unrestricted relative rotation between the first and second handle bodies 115, 135 and the protrusion 177 inhibit unrestricted relative translation between the first and second handle bodies 115, 135. Alternatively, the probe device 100 can have two or more sets of engagement members where a first one of the sets provides a first mode of motion control (e.g., anti-translation) and a second one of the sets provides a second mode of motion control (e.g., anti-rotation). An engagement member of the same general configuration as the engagement member 170 of the first handle body 115, but having a long narrow lever-like shape as opposed to a flat planar wall shape, and the mating engagement member 175 of the second handle body 135 are an example of such first one of the sets of engagement members that can be provided for engaging each other to inhibit only (or primarily) unrestricted relative translation between the first and second handle bodies 115, 135 in a given rotational direction Protruding flanges 179 of the first handle body 115 and the mating portion of the second handle body 135 are an example of such second one of the sets of engagement members that can be provided for engaging each other to inhibit only (or primarily) unrestricted relative rotation between the first and second handle bodies 115, 135 in a given rotational direction.

In one or more embodiments, it is preferred for the stimulation probe 120 to be detachable from the first handle body 115. To this end, the central portion 165 of the stimulation probe 120 is preferably translatably (e.g., slidably) disposed within the central passage 155 of the probe tube 140 such that the stimulation probe 120 can be translated along a length of the probe tube 140 for enabling the first handle assembly 105 to be detached from the second handle assembly 110. For enabling such translation, the longitudinal axis L2 of the probe tube 140 preferably extends coaxially along the longitudinal axis L1 of the probe stimulation probe 120 such that the stimulation probe is engaged within the central passage 155 of the probe tube 140 in a cannulated manner.

In use, a stimulation current (i.e., stimulation signal) is delivered from a neuromonitoring system through to a target tissue via a probe tip 180 of the stimulation probe 120. To this end (i.e., the stimulation signal being provided at the probe tip 180 of the stimulation probe 120 only), in one or more embodiments, the stimulation probe 120 is fully or partially made from an electrically-conductive material (e.g., stainless steel, nickel-plated polymer, of the like). To allow for the stimulation signal to be communicated between the probe tip 180 of the stimulation probe 120 and a signal communicating element 188 of the first handle assembly 105 (e.g., a conductive pin, wire or lead), electrically insulating material is preferably provided within the central passage 155 between the central portion 165 of the stimulation probe 120 and the probe tube 140. To this end, preferably, the electrically insulating material being provided within the central passage 155 between the central portion 165 of the stimulation probe 120 and the probe tube 140 comprises an interior surface of the central passage 155 comprises the electrically insulating material 163. For example, the probe tube 140 can be made from an electrically insulating material and/or the probe tube 140 can have electrical insulation (e.g., a layer of electrical insulation) provided thereon within the central passage 155 and/or electrical insulation (e.g., a layer of electrical insulation) can be provided on at least a portion of an exterior surface of the stimulation probe 120 at the central portion 165 thereof.

In one or more embodiments, the signal communicating element 188 provides an electrically-conductive interface with the stimulation probe 120 and mechanically secures the stimulation probe 120 in a fixed position relative to the first handle body 115. For example, the signal communicating element 188 can be a metal pin, tube or other structure that is press-fit, threaded, soldered or the like into an aperture within the stimulation probe 120 (i.e., extending from the stimulation probe 120 to an exterior surface of the handle assembly). Alternatively, the signal communicating element 188 can be in electrical contact with the stimulation probe 120 and a separate structure can provide for mechanical attachment of the stimulation probe 1220 to the first handle body 115.

In one or more embodiments, the signal communicating element 188 extends from electrical contact with the stimulation probe 120 to a connector access opening 189 in the handle body assembly. A terminal end of the signal communicating element 188 is accessible within the connector access opening 189. In use, a user connects a connector of a signal transmitting cable of a neuromonitoring system to the signal communicating element 188 for enabling electrical signal communication between the stimulation probe 120 and the neuromonitoring system. The terminal end of the signal communicating element 188 serves as a cable connector for allowing a user to connect the connecter of the signal transmitting cable thereto in a simple and convenient manner. To this end, the terminal end of the signal communicating element 188 is preferably in the form of a single or multi-contact electrical contact (e.g., an electrical pin, a multi-contact electrical plug or the like). In this regard, the signal communicating element 188 provides advantageous functionality in comparison to known types of probes that require direct electrical contact of a connector of the signal transmitting cable to the stimulation probe (e.g., via an electrically conductive mechanical clip).

In one or more embodiments, it is advantageous for different configurations of probe tips to be available to a surgeon during a surgical procedure. To this end, the first handle assembly 105 shown in FIG. 1 can be replaced with a different handle assembly that has a different configuration probe tip. Alternatively, the first handle body 115 can be configured such that the stimulation probe 120 can be detached from the first handle body 15 and replaced with a different stimulation probe having a different configuration probe tip. Similarly, as shown in FIG. 3, the second end portion 130 of the stimulation probe 120 can define a probe tip mount 190 including a probe tip engagement structure 192 (e.g., threaded interface) that engages a mating probe tip engagement structure 194 of the probe tip 180 thereby allowing the probe tip 180 to be detachably engaged with the probe tip mount 190. Examples of such different configuration probe tips include, but are not limited to, a probe tip with a pointed tip, a probe tip having a knife surface, a probe tip having a threaded exterior surface, a probe tip that comprises or consists of an implanted article (e.g., a bone screw or implement comprising a bone screw) and the like. In one or more embodiments, the stimulation probe 120 is a one-piece unit made from an electrically-conductive metal.

As previously disclosed, during spine surgery, inserting implanted articles (e.g., bone screws) into an anatomical structure (e.g., the bony portion of a spine) has risk of injury to the anatomical structure (e.g., the spinal cord and spine nerve roots). A probe device configured in accordance with embodiments of the present invention enables a surgeon to identify susceptible portions of a patient's anatomy in real-time (e.g., by monitoring nerve functions) and thereby more accurate and safely place an implanted article.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. A probe device, comprising:
a first handle assembly comprising a first handle body and a stimulation probe, wherein the stimulation probe has opposing end portions and wherein a first one of said opposing end portions of the stimulation probe is attached to the first handle body such that the stimulation probe protrudes away from the first handle body;
a second handle assembly comprising a second handle body and an probe tube, wherein the probe tube has opposing end portions, wherein a first one of said end portions of the probe tube is attached to the second handle body such that the probe tube protrudes away from the second handle body, wherein a central passage of the probe tube is accessible through a stimulation probe receiving passage of the second handle body, and wherein a central portion of the stimulation probe between said opposing end portion thereof is disposed within the central passage of the probe tube such that a first one of the opposing end portions of the stimulation probe is positioned adjacent to the second handle body and a second one of said opposing end portions of the stimulation probe is positioned adjacent to a second one of said opposing end portions of the probe tube, and wherein the central portion of the stimulation probe is translatably disposed within the central passage of the probe tube such that the stimulation probe can be translated along a length of the probe tube for enabling the first handle assembly to be detached from the second handle assembly.

2. The device of claim 1 wherein electrically insulating material is provided within the central passage between the central portion of the stimulation probe and the probe tube.

3. The device of claim 2 wherein the electrically insulating material being provided within the central passage includes an interior surface of the central passage comprising the electrically insulating material.

4. The device of claim 2 wherein the electrically insulating material comprises a layer of electrical insulation on at least a portion of an exterior surface of the stimulation probe at the central portion thereof.

5. The device of claim 1 wherein:
the stimulation probe and the probe tube are both elongated;
the stimulation probe has a round cross-sectional shape;
the probe tube has a round cross-sectional shape;
the stimulation probe is coaxially disposed within the central passage of the probe tube; and
a centerline longitudinal axis of the central passage of the probe tube extends along a straight reference axis.

6. The device of claim 1 wherein:
the first handle body and second handle body include mating anti-rotation engagement members that engage each other when the first handle body is in a seated position on the second handle body; and
unrestricted relative rotation movement between the first handle body and the second handle body in at least one rotational direction is inhibited when the mating anti-rotation engagement members thereof are engaged with each other.

7. The device of claim 6 wherein:
the first handle body and second handle body include mating anti-translation engagement members that engage each other when the first handle body is in a seated position on the second handle body; and
unrestricted relative axial movement between the first handle body and the second handle body is inhibited when the mating anti-translation engagement members thereof are engaged with each other.

8. The device of claim 7 wherein:
the stimulation probe and the probe tube are both elongated;
the stimulation probe has a round cross-sectional shape;
the stimulation probe tube has a round cross-sectional shape;
the stimulation probe is coaxially disposed within the central passage of the probe tube; and
a centerline longitudinal axis of the central passage of the probe tube extends along a straight reference axis.

9. The device of claim 1 wherein:
the first handle body and second handle body include mating engagement members that engage each other when the first handle body is in a seated position on the second handle body; and
at least one of unrestricted relative rotation movement in at least one rotational direction between the first handle body and the second handle body and unrestricted relative axial movement between the first handle body and the second handle body is inhibited when the mating engagement members thereof are engaged with each other.

10. The device of claim 1 wherein the second one of said opposing end portions of the stimulation probe defines one of a probe tip and a probe tip mount.

11. The device of claim 10, further comprising:
a probe tip;
wherein the second one of said opposing end portions of the stimulation probe defines a probe tip mount;
the probe tip mount includes a probe tip engagement structure; and
the probe tip is detachably engaged with the probe tip mount.

12. The device of claim 11 wherein:
the first handle body and the second handle body include mating engagement members that engage each other when the first handle body is in a seated position on the second handle body; and
unrestricted relative rotation movement in at least one rotational direction between the first handle body and the second handle body and unrestricted relative axial movement between the first handle body and the second handle body are both inhibited when the mating engagement members thereof are engaged with each other.

13. The device of claim 12 wherein electrically insulating material is provided within the central passage between the central portion of the stimulation probe and the probe tube thereof.

14. The device of claim 1, further comprising:
a signal communicating element in electrical contact with the stimulation probe, wherein the signal communicating element extends from the stimulation probe to a connector access opening in at least one of the first and second handles bodies.

15. A neuromonitoring probe device, comprising:
a handle including a lower handle body and an upper handle body;
a probe tube having opposing end portions, wherein a first one of said end portions of the probe tube is attached to the lower handle body such that the probe tube protrudes therefrom, wherein a central passage of the probe tube intersects a stimulation probe receiving passage of the lower handle body; and
a stimulation probe having opposing end portions and a central portion extending therebetween, wherein the stimulation probe is made from an electrically conductive material, wherein a first one of said opposing end portions is attached to the upper handle body, wherein the central portion of the stimulation probe is slideably disposed within the central passage of the probe tube for enabling the stimulation probe to be removed from therein by axially translating the stimulation probe relative to the probe tube, wherein a second one of said opposing end portion of the stimulation probe is located adjacent to a second one of said opposing end portions of the probe tube and wherein electrically insulating material is provided within the central passage between the central portion of the stimulation probe and the probe tube;
wherein the first handle body and the second handle body include mating engagement members that engage each other when the first handle body is in a seated position on the second handle body; and
wherein at least one of unrestricted relative rotation movement in at least one rotational direction between the first handle body and the second handle body and unrestricted relative axial movement between the first handle body and the second handle body is inhibited when the mating engagement members thereof are engaged with each other.

16. The neuromonitoring probe device of claim 15 wherein the electrically insulating material being provided within the central passage between the central portion of the stimulation probe and the probe tube thereof comprises at least one of an interior surface of the central passage comprising the electrically insulating material and electrical insulation being provided on at least a portion of an exterior surface of the stimulation probe at the central portion thereof.

17. The neuromonitoring probe device of claim 15 wherein:
the stimulation probe and the probe tube are both elongated;
the stimulation probe has a round cross-sectional shape;
the probe tube has a round cross-sectional shape;
the stimulation probe is coaxially disposed within the central passage of the probe tube; and
a centerline longitudinal axis of the central passage of the probe tube extends along a straight reference axis.

18. The neuromonitoring probe device of claim 15 wherein the second one of said opposing end portions of the stimulation probe defines one of a probe tip and a probe tip mount.

19. The neuromonitoring probe device of claim 18, further comprising:
a probe tip;
wherein the second one of said opposing end portions of the stimulation probe defines a probe tip mount;
the probe tip mount includes a probe tip engagement structure; and
the probe tip is detachably engaged with the probe tip mount.

20. The neuromonitoring probe device of claim 19 wherein the electrically insulating material being provided within the central passage between the central portion of the stimulation probe and the probe tube thereof comprises at least one of an interior surface of the central passage comprising the electrically insulating material and electrical insulation being provided on at least a portion of an exterior surface of the stimulation probe at the central portion thereof.
